SEQUENCE LISTING (3 sequences)

SEQ ID NO: 1 - DNA, 18 bp, ARTIFICIAL SEQUENCE
  RANDOM ARTIFICIAL SEQUENCE - NO SOURCE
gttttcccagtcacgacg

SEQ ID NO: 2 - DNA, 104 bp, ARTIFICIAL SEQUENCE
  RANDOM ARTIFICIAL SEQUENCE - NO SOURCE
agcgcgcgtaatacgactcactatagggcgaattgggtaccgggccccccctcgaggtcgacggtatcgataagcttgatatcgaattcctgcagcccggggga

SEQ ID NO: 3 - DNA, 88 bp, ARTIFICIAL SEQUENCE
  RANDOM ARTIFICIAL SEQUENCE - NO SOURCE
tccactagttctagagcggccgccaccgcggtggagctccagcttttgttccctttagtgagggttaattgcgcgcttggcgtaatca

(19) United States
(12) Patent Application Publication
(10) Pub. No.: US 2005/0032081 A1
(43) Pub. Date: Feb. 10, 2005

(54) Biomolecular coupling methods using 1,3-dipolar cycloaddition chemistry

(76) Inventors: Jingyue Ju, Englewood Cliffs, NJ (US); Tae Seok Seo, New York, NY (US)

Correspondence Address: Cooper & Dunham LLP, 1185 Avenue of the Americas, New York, NY, 10036, US

(21) Appl. No.: 10/735,081

(22) Filed: Dec. 11, 2003

Related U.S. Application Data
(60) Provisional application No. 60/433,440, filed on Dec. 13, 2002.

(51) Int. Cl. A61L 2/00; B05D 3/00; C12Q 1/68; G01N 33/53; C12M 1/34

(52) U.S. Cl. 435/6; 435/7.1; 435/287.2; 427/2.11

(57) ABSTRACT

This invention provides methods for covalently affixing a biomolecule to either a second molecule or a solid surface using 1,3-dipolar cycloaddition chemistry. This invention also provides related methods and compositions.

This application claims the benefit of copending U.S.

Provisional Application No. 60/433,440, filed Dec. 13, 2002, the contents of which are hereby incorporated by reference.

The invention disclosed herein was made with Government support under a grant from the National Science Foundation (Sensing and Imaging Initiative Grant 0097793). Accordingly, the U.S. Government has certain rights in this invention.

Throughout this application, various publications are referenced in parentheses by number. Full citations for these references may be found at the end of the specification immediately preceding the claims. The disclosures of these publications in their entireties are hereby incorporated by reference into this application to more fully describe the state of the art to which this invention pertains.

### BACKGROUND OF THE INVENTION

Synthetic oligonucleotides are the most important molecular tools for genomic research and biotechnology (1). Modified oligonucleotides are widely used as primers for DNA sequencing (2) and polymerase chain reaction (3), antisense agents for therapeutic applications (4), molecular beacons for detecting genetic mutations (5), and probes for measuring gene expression in DNA microarrays and gene chips (6). The modification of either the 3′- and 5′-termini or an internal position of the oligonucleotides with a primary alkyl amine group is a widely used method for introducing additional functional groups to DNA (7). Introduction of these functionalities to DNA can be achieved through the use of appropriate phosphoramidite reagents in solid phase synthesis. Once a unique functional group is incorporated into the DNA, the functional group can subsequently be conjugated to the desired molecule by a selective chemical reaction.

The succinimidyl ester of a fluorescent dye is widely used to couple with a primary amine group introduced to an oligonucleotide (8). However, the coupling reaction requires aqueous conditions that can hydrolyze the succinimidyl ester moiety. To overcome this difficulty, phosphoramidite derivatives of fluorescent dyes were used to directly couple with the oligonucleotide in the solid phase synthesis (9). However, if the functional group is labile to the basic deprotection conditions used in solid phase DNA synthesis, the direct phosphoramidite approach cannot be used. Thus, there is still a need to develop coupling chemistry with high stability and high yield to modify DNA and other biomolecules. To this end, chemoselective modification of protein and cell surfaces by the Staudinger ligation has been developed (10), and the Diels Alder reaction was also explored for the selective immobilization of proteins (11).

Ideal coupling functional groups (one on the DNA and the other on the molecule to be coupled) should be stable under aqueous reaction conditions. The coupling reaction should be highly chemoselective with a high yield, and the resulting linkage should be stable under biological conditions.

Recently, Sharpless et al. defined “click chemistry” as a set of powerful, highly reliable, and selective reactions for the rapid synthesis of useful new compounds and combinatorial libraries through heteroatom links (12). One of the click chemistry reactions involves the coupling between azides and alkynyl/alkynes to form the triazole version of Huisgen's [2+3] cycloaddition family (13). Mock et al. (14) discovered that cucurbituril could catalyze this 1,3-dipolar cycloaddition. This coupling chemistry was also used to form oligotriazoles and rotaxanes by Steinke et al. (15). The addition results in regioisomeric five-membered heterocycles (16). This 1,3-dipolar cycloaddition chemistry is very chemoselective, only occurring between alkynyl and azido functional groups with high yield. In addition, the resulting 1,2,3-triazoles are stable at aqueous conditions and high temperature.

### SUMMARY OF THE INVENTION

This invention provides a first method for covalently affixing a biomolecule to a second molecule comprising contacting a biomolecule having an azido group covalently and operably affixed thereto with a second molecule having an alkynyl group covalently and operably affixed thereto under conditions permitting a 1,3-dipolar cycloaddition reaction to occur between the azido and alkynyl groups, thereby covalently affixing the biomolecule to the second molecule.

This invention also provides a second method for covalently affixing a biomolecule to a second molecule comprising contacting a biomolecule having an alkynyl group covalently and operably affixed thereto with a second molecule having an azido group covalently and operably affixed thereto under conditions permitting a 1,3-dipolar cycloaddition reaction to occur between the alkynyl and azido groups, thereby covalently affixing the biomolecule to the second molecule.

This invention also provides a first method for covalently affixing a biomolecule to a solid surface comprising contacting a biomolecule having an azido group covalently and operably affixed thereto with a solid surface having an alkynyl group operably affixed thereto under conditions permitting a 1,3-dipolar cycloaddition reaction to occur between the azido and alkynyl groups, thereby covalently affixing the biomolecule to the solid surface.

This invention further provides a second method for covalently affixing a biomolecule to a solid surface comprising contacting a biomolecule having an alkynyl group covalently and operably affixed thereto with a solid surface having an azido group operably affixed thereto under conditions permitting a 1,3-dipolar cycloaddition reaction to occur between the alkynyl and azido groups, thereby covalently affixing the biomolecule to the solid surface.

This invention further provides a biomolecule having either an azido group or an alkynyl group covalently and operably affixed thereto.

This invention further provides a solid surface having an azido group or an alkynyl group operably affixed thereto.

This invention provides a biomolecule covalently affixed to a second molecule via one of the instant methods.

This invention further provides a biomolecule covalently affixed to a solid surface via one of the instant methods.

This invention further provides a biomolecule covalently affixed to a second molecule via a 1,2,3-triazole ring.

Finally, this invention further provides a biomolecule covalently affixed to a solid surface via a 1,2,3-triazole ring.

### BRIEF DESCRIPTION OF THE FIGURES

FIG. 1: Scheme for synthesizing an oligonucleotide labeled by an azido group at the 5′ end.

FIG. 2: MALDI-TOF mass spectrum of structure 2 of FIG. 1.

FIG. 3: Scheme showing 1,3-dipolar cycloaddition between alkynyl-FAM and azido-labeled DNA.

FIG. 4: MALDI-TOF MS spectrum of structures 4 and 5 of FIG. 3.

FIG. 5: Electropherogram of the DNA sequencing fragments generated with structures 4 and 5.

FIG. 6: Immobilization of a polypeptide on a solid surface.

FIG. 7: Immobilization of a polypeptide on a solid surface.

FIG. 8: Immobilization of a polysaccharide on a solid surface.

FIG. 9: Immobilization of protein on a solid surface.

FIG. 10: Immobilization of an oligonucleotide on a solid surface.

FIG. 11: Immobilization of DNA on a glass surface in the presence of Cu(I) Catalyst.

### DETAILED DESCRIPTION OF THE INVENTION

Definitions

As used herein, and unless stated otherwise, each of the following terms shall have the definition set forth below.

“Antibody” shall include, by way of example, both naturally occurring and non-naturally occurring antibodies. Specifically, this term includes polyclonal and monoclonal antibodies, and fragments thereof. Furthermore, this term includes chimeric antibodies and wholly synthetic antibodies, and fragments thereof.

“Biomolecule” shall mean a molecule occurring in a living system or non-naturally occurring analogs thereof, including, for example, amino acids, peptides, oligopeptides, polypeptides, proteins, nucleotides, oligonucleotides, polynucleotides, nucleic acids, DNA, RNA, lipids, enzymes, receptors and receptor ligand-binding portions thereof.

“Carbohydrate” shall mean an aldehyde or ketone derivative of a polyhydroxy alcohol that is synthesized by living cells, and includes monosaccharides, disaccharides, oligosaccharides, and polysaccharides synthesized from saccharide monomers.

“Covalently affixing” shall mean the joining of two moieties, via a covalent bond.

“Lipid” shall mean a hydrophobic organic molecule including, but not limited to, a steroid, a fat, a fatty acid, or a phospholipid.

“Nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Operably affixed” in reference to an azido group or an alkynyl group shall mean that the group is affixed to a molecule or surface in such a way as to permit the azido or alkynyl group to undergo a 1,3-dipolar cycloaddition with an alkynyl or azido group, respectively, on a different molecule or surface, as applicable.

“Rₙ”, in an embodiment where the biomolecule is a peptide, can be a side chain of n amino acids. Each repeating unit is, for example, one of 20 amino acids or their analogues, and shall include e.g. Glycine, Alanine, Valine, Leucine, Isoleucine, Proline, Phenylalanine, Tyrosine, Tryptophan, Serine, Threonine, Cysteine, Methionine, Asparagine, Glutamine, Aspartate, Glutamate, Lysine, Arginine, Histidine. Lysine, Arginine, Serine, Cysteine, or Threonine is preferred as the carboxyl-terminal residue. n can be, for example, 1-500.

In an embodiment where the biomolecule is a sugar, the azido or alkynyl functional group is located at the terminal sugar ring.

In an embodiment where the biomolecule is an oligonucleotide, R is a hydrogen for DNA and a hydroxyl group for RNA, and N is, for example, 1-200. “B” groups are heterocyclic ring systems called bases. The principal bases are adenine, guanine, cytosine, thymine, and uracil.

In an embodiment where the biomolecule is a protein, for example, an enzyme, antigen, or antibody, the positions of the azido and the alkynyl functional groups are easily interchangeable.

In the instant embodiments, “X” can be, for example, an aliphatic or aliphatic-substituted derivative, aryl or aryl-substituted group, electron-withdrawing functional group or electron-releasing group. An aliphatic chain shall include, for example, a lower alkyl group, in particular C₁-C₅ alkyl, which is unsubstituted or mono- or polysubstituted, e.g. methyl, ethyl, n-propyl, isopropyl, n-butyl, isobutyl, tert-butyl, or n-pentyl. An aryl or aryl-substituted group shall include, for example, a phenyl, or an o-, m-, p-substituted phenyl, e.g. p-methylphenyl, p-chlorophenyl, p-nitrophenyl group. An electron-withdrawing functional group shall include, for example, an alkoxy substituted alkyl, e.g. diethoxymethyl, or halogenated carbon substituent, e.g. chloromethyl, trifluoromethyl, or an alkyl ester, e.g. methyl ester, ethyl ester, or a ketone derivative, e.g. methyl ketone, ethyl ketone, aryl ketone, or a substituted sulfonyl derivative, e.g. arenesulfonyl, or substituted phosphinyl, e.g. diphenylphosphinyl, diethoxyphosphinyl. An electron-releasing group shall include, for example, an alkoxy group, e.g. methoxy, ethoxy, or an alkylamino group, e.g. diethylamino, phenylmethylamino.

Embodiments of the Invention

This invention provides a first method for covalently affixing a biomolecule to a second molecule comprising contacting a biomolecule having an azido group covalently and operably affixed thereto with a second molecule having an alkynyl group covalently and operably affixed thereto under conditions permitting a 1,3-dipolar cycloaddition reaction to occur between the azido and alkynyl groups, thereby covalently affixing the biomolecule to the second molecule.

This invention also provides a second method for covalently affixing a biomolecule to a second molecule comprising contacting a biomolecule having an alkynyl group covalently and operably affixed thereto with a second molecule having an azido group covalently and operably affixed thereto under conditions permitting a 1,3-dipolar cycloaddition reaction to occur between the alkynyl and azido groups, thereby covalently affixing the biomolecule to the second molecule.

In the first and second methods the biomolecule can be, for example, a nucleic acid, a protein, a peptide, a carbohydrate, or a lipid. In one embodiment the biomolecule is DNA, an antibody, an enzyme, or a receptor or a ligand-binding portion thereof. In other embodiments, the biomolecule can be a nucleotide, an oligonucleotide, a polynucleotide, a lipid, a lipid derivative, an amino acid, a peptide, an oligopeptide, a polypeptide, a protein, a monosaccharide, a disaccharide, an oligosaccharide, or a polysaccharide.

Also, in the first and second methods, the second molecule can be, for example, a biomolecule, a fluorescent label, a radiolabeled molecule, a dye, a chromophore, an affinity label, an antibody, biotin, streptavidin, a metabolite, a mass tag, or a dextran. In other embodiments, the biomolecule can be a nucleotide, an oligonucleotide, a polynucleotide, a lipid, a lipid derivative, an amino acid, a peptide, an oligopeptide, a polypeptide, a protein, a monosaccharide, a disaccharide, an oligosaccharide, or a polysaccharide.

In one embodiment of the first and second methods, the biomolecule is immobilized. In another embodiment, the second molecule is immobilized. In a further embodiment, neither the biomolecule nor the second molecule is immobilized.

Conditions permitting a 1,3-dipolar cycloaddition reaction to occur are known, and can comprise for example, the application of heat, contacting at room temperature, and contacting at 4° C. Optionally, the contacting is performed in the presence of an agent which catalyzes a 1,3-dipolar cycloaddition reaction. In the absence of the catalyst the reaction is carried about within the temperature range 50° C. to 150° C., and more usually at between 70° C. to 100° C. The molar ratio of cataylyst:alkynyl group:azido group is from 0:1:1 to 2:1:100, and preferably 1:1:0.5. The reaction is carried out in the aqueous phase or aqueous/water-soluble organic mixture such as water/dimethylformamide or water/methyl sulfoxide as the solvent system. The molar ratio between the alkynyl group and the azido group is from 1:1 to 1:100. In the presence of a catalyst, such as a Cu(I) catalyst, the reaction may be performed at room temperature.

This invention also provides a first method for covalently affixing a biomolecule to a solid surface comprising contacting a biomolecule having an azido group covalently and operably affixed thereto with a solid surface having an alkynyl group operably affixed thereto under conditions permitting a 1,3-dipolar cycloaddition reaction to occur between the azido and alkynyl groups, thereby covalently affixing the biomolecule to the solid surface.

This invention further provides a second method for covalently affixing a biomolecule to a solid surface comprising contacting a biomolecule having an alkynyl group covalently and operably affixed thereto with a solid surface having an azido group operably affixed thereto under conditions permitting a 1,3-dipolar cycloaddition reaction to occur between the alkynyl and azido groups, thereby covalently affixing the biomolecule to the solid surface.

In the first and second surface-related methods, the embodiments of biomolecules and reaction conditions are the same as those set forth above in connection with the first and second methods for affixing a biomolecule to a second molecule.

In the first and second surface-related methods, the solid surface can be, for example, glass, silica, diamond, quartz, gold, silver, metal, polypropylene, or plastic. In the preferred embodiment the solid surface is silica. The solid surface can be present, for example, on a bead, a chip, a wafer, a filter, a fiber, a porous media, or a column.

This invention further provides a biomolecule having either an azido group or an alkynyl group covalently and operably affixed thereto. This biomolecule can be, for example, a nucleic acid, a protein, a peptide, a carbohydrate, or a lipid. Preferably, the biomolecule is DNA.

This invention further provides a solid surface having an azido group or an alkynyl group operably affixed thereto. This solid surface of can be, for example, glass, silica, diamond, quartz, gold, silver, metal, polypropylene, or plastic. The solid surface can be, for example, present on a bead, a chip, a wafer, a filter, a fiber, a porous media, or a column. Preferably, the solid surface is a silica surface. Preferably, the silica surface is part of a chip.

This invention provides a biomolecule covalently affixed to a second molecule via one of the instant methods.

This invention further provides a biomolecule covalently affixed to a solid surface via one of the instant methods.

This invention further provides a DNA molecule covalently attached to a glass surface via one of the instant methods.

This invention further provides a biomolecule covalently affixed to a second molecule via a 1,2,3-triazole ring.

Finally, this invention further provides a biomolecule covalently affixed to a solid surface via a 1,2,3-triazole ring.

This invention will be better understood by reference to the Experimental Details which follow, but those skilled in the art will readily appreciate that the specific experiments detailed are only illustrative of the invention as described more fully in the claims which follow thereafter.

Experimental Details

Here we disclose using highly chemoselective, high yield click chemistry to couple biomolecules to other components, including solid supports. This optimized click chemistry has applications in bio-conjugation fields including DNA covalent attachment on a chip, chemoselective protein modification, and immunoassays.

### EXAMPLE 1

We explored the use of the “click chemistry” 1,3-dipolar cycloaddition reaction to couple a fluorophore to DNA. We show the synthesis of fluorescent single-stranded DNA (ssDNA) using the “click chemistry”, and the application of the fluorescent ssDNA as a primer in the Sanger dideoxy chain termination reaction (17) to produce DNA sequencing fragments.

Click chemistry 1,3-dipolar cycloaddition between alkynyl 6-carboxyfluorescein (FAM) and azido-labeled single-stranded (ss) DNA was carried out under aqueous conditions to produce FAM-labeled ssDNA in quantitative yield. The FAM-labeled ssDNA was successfully used to produce DNA sequencing products with singe base resolution in a capillary electrophoresis DNA sequencer with laser-induced fluorescence detection.

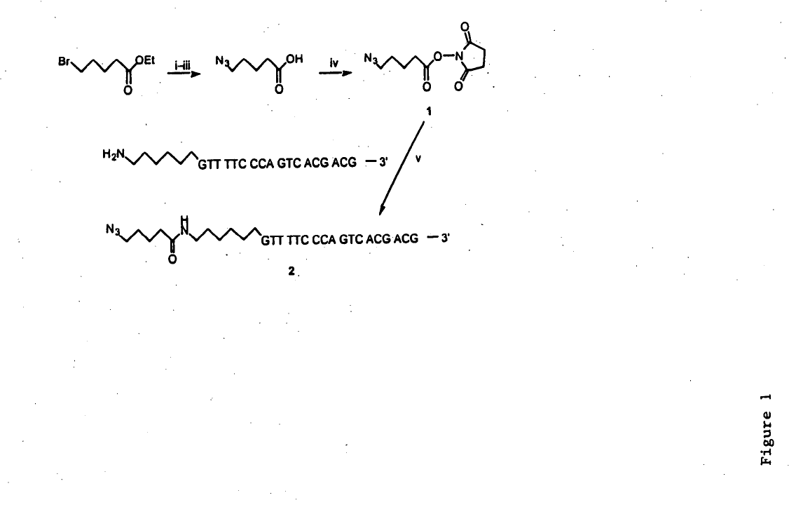

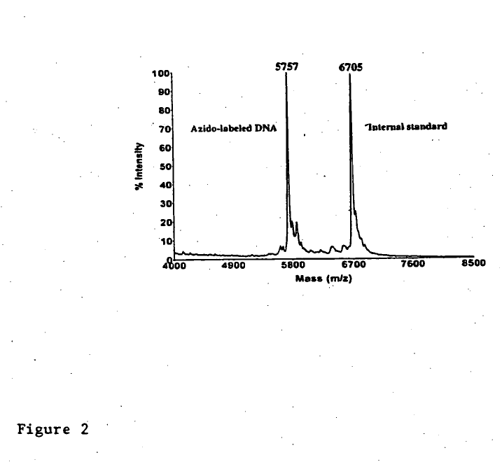

Initially, we synthesized an oligonucleotide labeled by an azido group at the 5′ end as shown in FIG. 1. 5-Azidovaleric acid was synthesized according to the literature (18) and activated as N-succinimidyl ester “1” (87%). The oligonucleotide 5′-amino-GTT TTC CCA GTC ACG ACG-3′ (M13-40 universal forward sequencing primer) was reacted with excess succinimidyl 5-azidovalerate “1” to produce the azido-labeled DNA “2” (see FIG. 1). After size-exclusion chromatography to remove excess starting material 1 and desalting with an oligonucleotide purification cartridge, the product was analyzed with matrix-assisted laser desorption/ionization time-of-flight mass spectrometry (MALDI-TOF MS). FIG. 2 shows the MALDI-TOF MS spectrum of the isolated product, with a single major peak at 5757 Da that matched very well with the calculated value of 5758 Da for the azido-DNA 2. This indicates that the starting material amino-DNA was quantitatively converted to the azido-DNA 2 (coupling yield ˜96%).

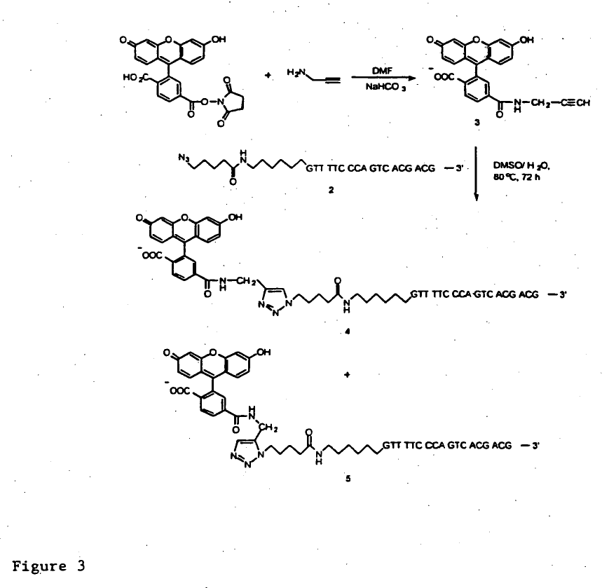

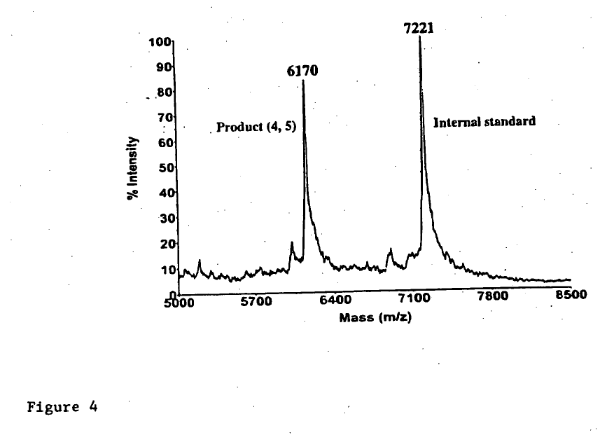

We then synthesized an alkynyl 6-carboxyfluorescein (FAM) “31” by reacting propargylamine with 6-carboxyfluorescein-NHS ester (see FIG. 3). The “click chemistry” 1,3-dipolar cycloaddition between the alkynyl-FAM and the azido-labeled DNA 2 was carried out at 80° C. in aqueous condition to produce the FAM-labeled DNAs “4” and “5” (see FIG. 3). After the reaction, excess alkynyl-FAM was removed by size-exclusion chromatography and the resulting FAM labeled DNAs “4” and “5” were desalted with an oligonucleotide purification cartridge. We characterized the products “4” and “5” by measuring their UV/Vis absorption and MALDI-TOF MS spectra. Characteristic peaks with maxima of 500 nm (FAM) and 260 nm (DNA) were obtained by UV/Vis measurement. The MALDI-TOF MS spectrum of “4” and “5”, is shown in FIG. 4. The mass peak of the azido-labeled DNA (5758 Da) almost completely disappeared and a single major peak at 6170 Da corresponding to the cycloaddition reaction product (4 and 5, theoretical mass value of 6169 Da) was obtained with an isolated yield of 91%.

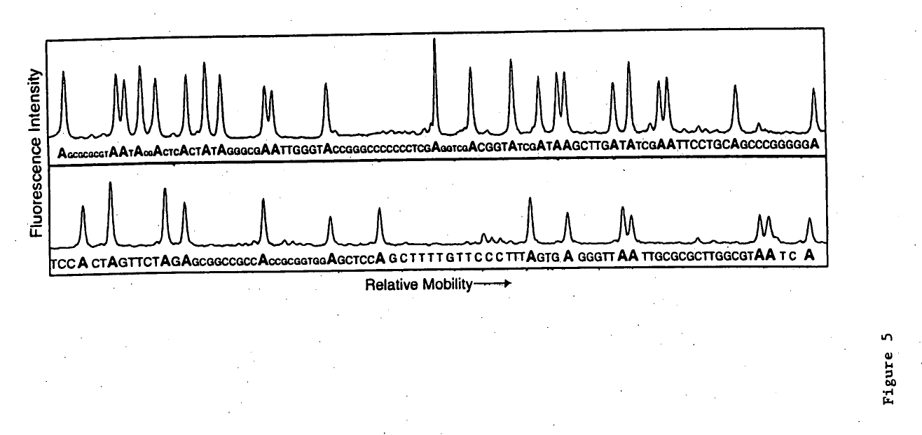

To demonstrate the utility of the FAM-labeled oligonucleotide “4” and “5” constructed by click chemistry for DNA analysis, we used the oligonucleotides in the Sanger dideoxy chain termination method to produce DNA sequencing fragments terminated by biotinylated dideoxyadenine triphosphate (ddATP-Biotin) using PCR amplified DNA as a template. Solid-phase capture using streptavidin-coated magnetic beads allows the isolation of pure DNA extension fragments free from false terminations (19). These DNA fragments were analyzed by a capillary array electrophoresis (CAE) system (20) and resolved at single base pair (bp) resolution to produce an electropherogram as shown in FIG. 5. The peaks represent the FAM fluorescence emission from each DNA fragment that was extended from “4” and “5”, and terminated by ddATP. This “A” sequencing ladder shown in FIG. 5 matched exactly with the sequence of the DNA template.

Without further purification by gel electrophoresis and HPLC that are required for conventional fluorescent oligonucleotide synthesis, the primer synthesized by the click chemistry can be used directly to produce DNA sequencing products with singe base resolution in a capillary electrophoresis DNA sequencer with laser induced fluorescence detection. A reduced reaction time can be achieved by attaching an electron withdrawing functional group at the end of the triple bond (12).

### EXAMPLE 2

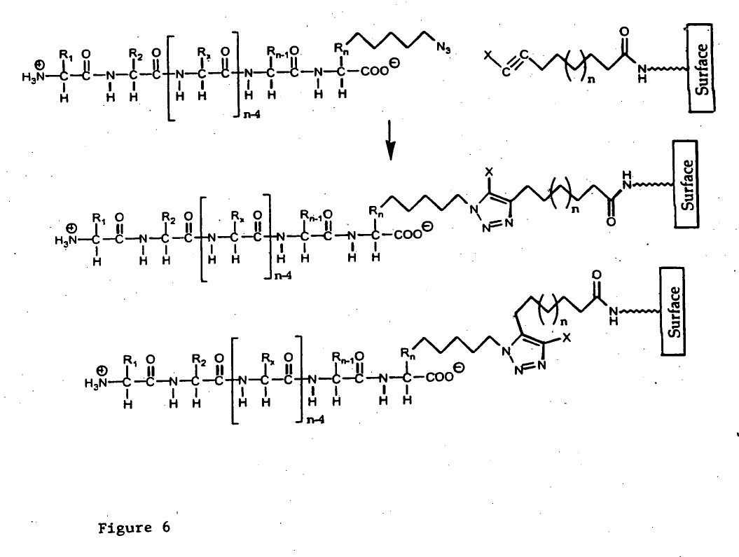

Peptides can be similarly bonded to other biomolecules or solid surfaces. FIG. 6 shows the immobilization of a polypeptide on a solid surface by 1,3-dipolar cycloaddition reaction. The polypeptide is labeled with an azido group at the carboxyl-terminal residue, while the solid surface is modified by a heterobifunctional linker which produces a substituted alkynyl group at the end. After the 1,3-dipolar cycloadditon between the azido and the alkynyl group, the polypeptide is covalently attached to the surface via a stable 1,2,3-triazole linkage.

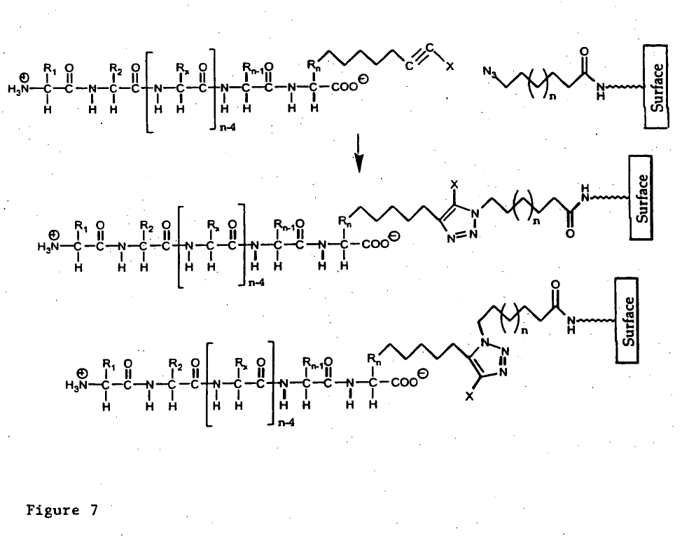

The positions of the azido and the alkynyl functional groups are easily interchangeable. FIG. 7 shows the scheme for the immobilization of a polypeptide on a solid surface by 1,3-dipolar cycloaddition reaction. The polypeptide is labeled with a substituted alkynyl group at the carboxyl-terminal residue, while the solid surface is modified by a heterobifunctional linker which produces an azido group at the end. After the 1,3-dipolar cycloaddition between the azido and the alkynyl group, the polypeptide is covalently attached to the surface via a stable 1,2,3-triazole linkage.

The 1,3-dipolar cycloadditon reaction is controlled either thermodynamically at high temperature, or catalytically at room temperature with cucurbituril (21). In the absence of the catalyst, the reaction is carried about within the temperature range 50° C. to 150° C., and more usually at between 70° C. to 100° C.

Without the catalyst, the reaction takes from 5 hours to 7 days depending on the substituents referred to as “X” in FIGS. 6 and 7. The molar ratio of cataylyst:alkynyl group:azido group is from 0:1:1 to 2:1:100, and preferably 1:1:0.5. The reaction is carried out in the aqueous phase or aqueous/water-soluble organic mixture such as water/dimethylformamide or water/methyl sulfoxide as the solvent system.

### EXAMPLE 3

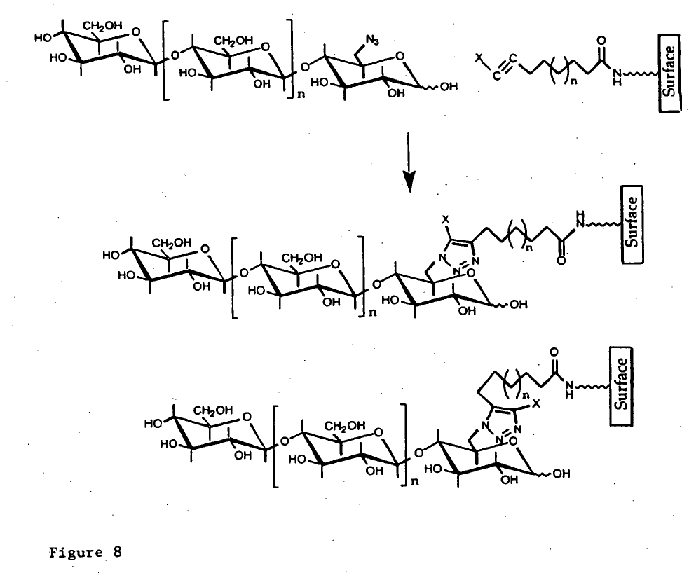

Sugars can be similarly bonded to other biomolecules or solid surfaces. FIG. 8 shows a scheme for the immobilization of a polysaccharide on a solid surface by 1,3-dipolar cycloaddition reaction. The polysaccharide is labeled with an azido group at the terminal sugar ring, while the solid surface is modified by a heterobifunctional linker which produces a substituted alkynyl group at the end. After the 1,3-dipolar cycloaddition between the azido and the alkynyl group, the polysaccharide is covalently attached to the surface via a stable 1,2,3-triazole linkage. The positions of the azido and the alkynyl functional groups are interchangeable as similarly shown in FIGS. 6 and 7.

The 1,3-dipolar cycloadditon reaction is controlled either thermodynamically at high temperature, or catalytically at room temperature with cucurbituril (21). In the absence of the catalyst the reaction is carried about within the temperature range 50° C. to 150° C., and more usually at between 70° C. to 100° C. The reaction takes from 5 hours to 7 days depending on the substituents referred to as “X” in FIGS. 6-9. The molar ratio of catalyst:alkynyl group:azido group is from 0:1:1 to 2:1:100, and preferably 1:1:0.5. The reaction is carried out in the aqueous phase or aqueous/water-soluble organic mixture such as water/dimethylformamide or water/methyl sulfoxide as the solvent system.

### EXAMPLE 4

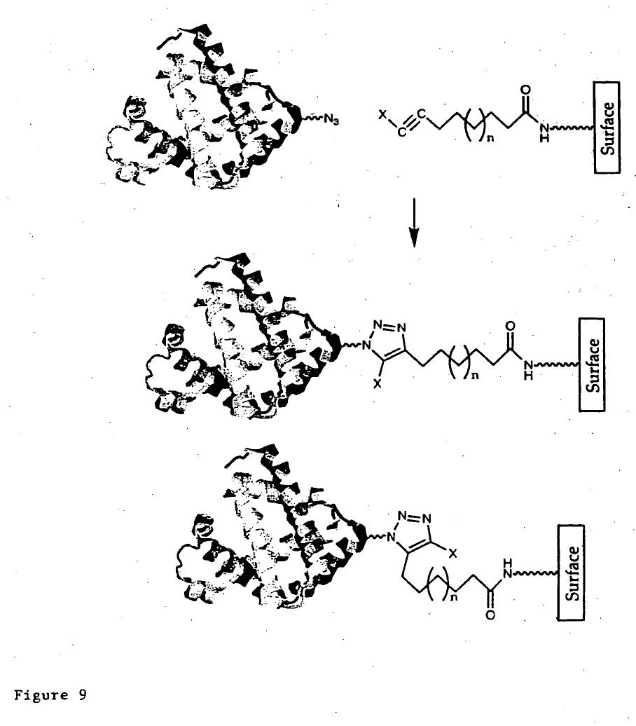

Proteins can be similarly bonded to other biomolecules or solid surfaces. FIG. 9 shows a scheme for the immobilization of a protein on a solid surface by 1,3-dipolar cycloaddition reaction. The protein is labeled with an azido group, while the solid surface is modified by a heterobifunctional linker which produces a substituted alkynyl group at the end. After the 1,3-dipolar cycloaddition between the azido and the alkynyl group, the protein is covalently attached to the surface via a stable 1,2,3-triazole linkage. The positions of the azido and the alkynyl functional groups are interchangeable as similarly shown in FIGS. 6 and 7.

The 1,3-dipolar cycloadditon reaction is controlled either thermodynamically at high temperature, or catalytically at room temperature with cucurbituril (21). In the: absence of the catalyst the reaction is carried about within the temperature range 50° C. to 150° C., and more usually at between 70° C. to 100° C. The reaction takes from 5 hours to 7 days depending on the substituents referred to as “X” in FIGS. 6-9. The molar ratio of catalyst:alkynyl group:azido group is from 0:1:1 to 2:1:100, and preferably 1:1:0.5. The reaction is carried out in the aqueous phase or aqueous/water-soluble organic mixture such as water/dimethylformamide or water/methyl sulfoxide as the solvent system.

### EXAMPLE 5

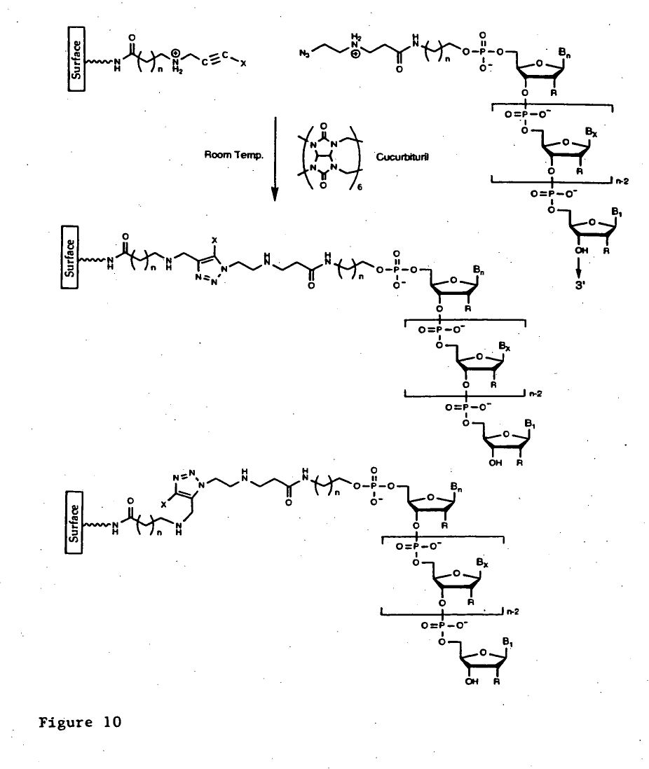

Nucleotides, oligonucleotides and polynucleotides can be similarly bonded to other biomolecules or solid surfaces. FIG. 10 shows a scheme for the immobilization of an oligonucleotide on a solid surface by 1,3-dipolar cycloaddition reaction. The oligonucleotide is labeled with an azido group at the 5′ end, while the solid surface is modified by a heterobifunctional linker which produces a substituted alkynyl group as the terminal functional group. After the 1,3-dipolar cycloaddition between the azido and the alkynyl group, the oligonucleotide is covalently attached to the surface via a stable 1,2,3-triazole linkage. The positions of the azido and the alkynyl functional groups are interchangeable as similarly shown in FIGS. 6 and 7.

The 1,3-dipolar cycloadditon reaction is controlled either thermodynamically at high temperature, or catalytically at room temperature with cucurbituril (21). In the absence of the catalyst the reaction is carried about within the temperature range 50° C. to 150° C., and more usually at between 70° C. to 100° C. The molar ratio of catalyst:alkynyl group:azido group is from 0:1:1 to 2:1:100, and preferably 1:1:0.5. The reaction is carried out in the aqueous phase or aqueous/water-soluble organic mixture such as water/dimethylformamide or water/methyl sulfoxide as the solvent system.

### EXAMPLE 6

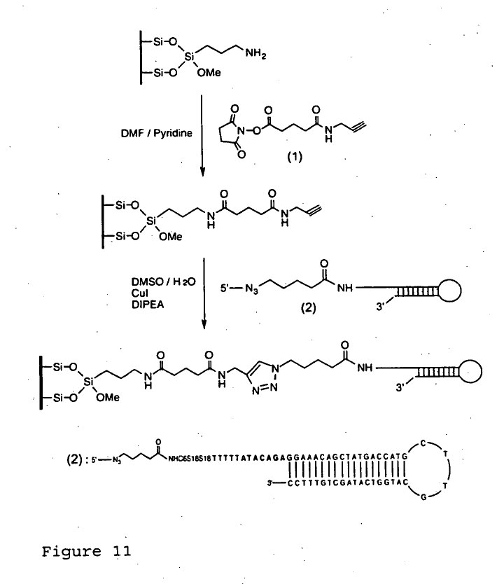

DNA can be bonded to solid surfaces such as glass at room temperature in the presence of a suitable catalyst. FIG. 11 shows a scheme for the immobilization of a DNA on a glass surface by 1,3-dipolar cycloaddition reaction in the presence of a Cu(I) catalyst. The DNA is labeled with an azido group at the 5′ end, while the glass surface is modified by an alkynyl group. After the 1,3-dipolar cycloaddition between the azido and the alkynyl group in the presence of a Cu(I) catalyst at room temperature, the DNA is covalently attached to the surface via a stable 1,2,3-triazole linkage. The positions of the azido and the alkynyl functional groups are interchangeable.

Materials and Methods for Examples 1-6

Materials and General Procedures. The amino-C6-M13 (−40) forward primer (18 mer) and the internal mass standard oligonucleotides were commercially available and purified by HPLC. The 1H and ¹³C NMR spectra were recorded on 400 MHz and 300 MHz NMR spectroscopic instruments, respectively. The high-resolution mass spectra (HRMS) were obtained under fast atom bombardment (FAB) conditions. UV-Vis spectra of the DNA samples were recorded in acetonitrile/water (1:1 volume ratio) at room temperature using quartz cells with path lengths of 1.0 cm.

Synthesis of succinimidyl 5-azidovalerate. 5-azidovaleric acid was synthesized according to the published procedure (18). 500 mg (2.61 mmol) of 1-(3-dimethylaminopropyl)-3-ethylcarbodiimide hydrochloride (EDC) was added to a suspension of 358 mg (2.50 mmol) of 5-azidovaleric acid and 300 mg (2.61 mmol) of N-hydroxysuccinimide in CH₂Cl₂ (20 mL) at room temperature and stirred for 7 h, followed by the addition of H₂O. The separated CH₂Cl₂ phase was washed with H₂O and brine solution, then dried over Na₂SO₄ and evaporated to yield 520 mg (87%) of succinimidyl 5-azidovalerate as a pale yellow liquid. IR (thin film) v 2100, 1640 cm-1; ¹H NMR (CDCl₃) δ 3.31 (t, 2H, J=6.6 Hz), 2.81 (s, 4H), 2.63 (t, 2H, J=7.1 Hz), 1.86-1.68 (m, 4H); ¹³C NMR (CDCl₃) δ 169.1, 168.2, 50.8, 30.4, 27.8, 25.5, 21.8; HRMS (FAB⁺) Cald. for C₉H₁₃O₄N₄, 241.0937 (M⁺H⁺); found, 241.0948.

Synthesis of an azido-labeled DNA. To incorporate the azido group at the 5′-end of the oligonucleotide, 10 nmol of amino-modified oligonucleotide in 40 μL of 0.25 M Na₂CO₃/NaHCO₃ buffer (pH 9.0) was incubated for 12 hours at room temperature with 10 μmol of succinimidyl 5-azidovalerate 1 in 12 μL of dimethyl sulfoxide. Unreacted succinimidyl 5-azidovalerate was removed by size-exclusion chromatography on a PD-10 column and the resulting azido-labeled DNA was desalted with an oligonucleotide purification cartridge. The concentration of the collected azido-labeled DNA was measured by an UV/Vis spectrophotometer and the isolated yield was 96%.

Synthesis of 6-carboxyfluorescein-propargylamide (Alkynyl FAM). A solution of 3.4 μL (0.05 mmol) of propargylamine in DMF (0.5 mL) was added to a solution of 11 mg (0.023 mmol) of 6-carboxyfluorescein-NHS ester in DMF (0.5 mL) and 0.1 M NaHCO₃ solution (0.1 mL). After 5 h of stirring at room temperature, the solvent was removed under vacuum and the crude mixture was purified by a silica gel TLC plate (MeOH/CHCl₃, 1:9) to give 8.0 mg (85%) of alkynyl FAM (Rf=0.45) as a red oil. ¹H NMR (Methanol-d4) δ 8.01 (s, 2H), 7.60 (s, 1H), 6.94 (d, 2H, J=9.1 Hz), 6.58-6.53 (m, 4H), 4.05 (d, 2H, J=2.4 Hz), 2.50 (t, 1H, J=2.2 Hz); ¹³C NMR (Methanol-d4) δ 175.3, 168.3, 158.5, 146.7, 136.9, 132.2, 129.9, 129.5, 128.7, 122.2, 121.0, 114.5, 104.0, 80.5, 72.2, 30.0; HRMS (FAB⁺) Cald. for C₂₄H₁₆O₆N, 414.0978 (M+2H+); found, 414.0997.

Synthesis of fluorescent DNA by click chemistry. 3.93 nmol of the azido-oligonucleotide in 120 μL water was reacted with a 150-fold excess of alkynyl FAM in 36 μL DMSO at 80° C. for 72 h. Unreacted dye was removed by size-exclusion chromatography on a PD-10 column. The resulting fluorescent DNA was then desalted with an oligonucleotide purification cartridge, and the concentration was measured by an UV/Vis spectrophotometer. The isolated yield of 4 and 5 was 91%.

DNA immobilization on a glass surface using the 1,3-dipolar cycloaddition coupling chemistry. The amino-modified glass (Sigma) surface was cleaned by immersion into a basic solution (dimethylformamide (DMF)/N,N-diisopropyl-ethylamine (DIPEA) 90/10 v/v) for 1 h, sonicated for 5 min, washed with DMF and ethanol, and then dried under air. The precleaned glass surface was functionalized by immersing it into the terminal alkyne crosslinker solution (20 mM of succinimidyl N-propargyl glutariamidate in DMF/pyridine (90/10 v/v)) for 5 h at room temperature. After sonication for 5 min, the glass surface was washed with DMF and ethanol and dried under air. Azido-labeled DNA was dissolved in DMSO/H₂O (1/2 v/v) to obtain a 20 μM solution. This DNA solution was then spotted onto the alkynyl-functionalized glass surface in the form of 4-μL drops, followed by the addition of Cu(I) (400 pmol, 5 eq.) and DIPEA (400 pmol, 5 eq.) solution. The glass slide was incubated in a humid chamber at room temperature for 12 h, then washed with dH₂O, and SPSC buffer (0.25 M sodium phosphate, 2.5 M NaCl, pH 6.5) extensively for 1 h to remove nonspecifically bound DNAs (28), and finally rinsed with dH₂O and ethanol. Atomic force microscopy (AFM) and water contact angle measurement were used for the characterization of the change on the surface after each step in the immobilization process.

Mass spectrum of DNA. Mass measurement of oligonucleotides was performed using a MALDI-TOF mass spectrometer. 30 pmol of the DNA product was mixed with 10 pmol of the internal mass standard and the mixture was suspended in 2 μL of 3-hydroxypicolinic acid matrix solution. 0.5 μL of this mixture was spotted on a stainless steel sample plate, air-dried and analyzed.

The measurement was taken using a positive ion mode with 25 kV accelerating voltage, 94% grid voltage and a 350 ns delay time.

PCR amplification of template. A PCR DNA product amplified from a pBluescript II SK(+) phagemid vector was used as a sequencing template as it has a binding site for M13-40 universal primer. Amplification was carried out using the M13-40 universal forward and reverse primers in a 20 μL reaction, which contained 1× ACCUTAQ LA Reaction Buffer, 25 pmol of each dNTP, 40 pmol of each primer, 0.5 unit of Jumpstart Red ACCUTAQ LA DNA Polymerase and 100 ng of the phagemid template. The reaction was performed in a DNA thermal cycler using an initial activation step of 96° C. for 1 minute. This was followed by 30 cycles of 94° C. for 30 seconds, 50° C. for 30 seconds, and 72° C. for 2 minutes. At the end of the PCR reaction, 20 μL of an enzymatic mixture containing 5 units of shrimp alkaline phosphatase (SAP), 4 μL of 10×SAP buffer, 6 units of E. Coli exonuclease 1 and 10 μL water was added to the PCR reaction to degrade the excess primers and dNTPs. The reaction mixture was incubated at 37° C. for 90 min before the enzymes were heat-inactivated at 72° C. for 30 min.

Generation and Detection of Sanger DNA Sequencing Fragments. A primer extension reaction was performed using the FAM-labeled primer “4” and “5” and the above PCR product. A 30 μL reaction mixture was made, consisting of 2.22 nmol of each dNTP, 37 pmol of Biotin-11-ddATP, 20 pmol of primer, 9 units of Thermo Sequenase DNA polymerase, 1× Thermo Sequenase Reaction Buffer and 20 μL of PCR product. The reaction consisted of 30 cycles of 94° C. for 20 seconds, 50° C. for 20 seconds and 60° C. for 90 seconds. Correctly terminated DNA fragments by Biotin-11-ddATP were purified from other reaction components using solid phase capture according to the published method (19). The fluorescent DNA fragments in 8 μL of formamide were electrokinetically injected at 3 kV into a capillary filled with linear polyacrylamide (LPA) gel in a capillary array fluorescent DNA sequencer, and then separated at 8 kV in LPA buffer to produce a fluorescence electropherogram.

### REFERENCES

(1) Caruthers, M. H., Science 1985, 230, p281.
(2) (a) Smith, L. M., Sanders, J. Z., Kaiser, R. J., Hughes, P., Dodd, C., Connell, C. R., Heiner, C., Kent, S. B. H., Hood, L. E., Nature 1986, 321, p674. (b) Ju, J., Ruan, C., Fuller, C. W., Glazer, A. N., and Mathies, R. A. Proc. Natl. Acad. Sci.

U.S.A. 1995, 92, p4347.
(3) Mullis, K. B., and Faloona, F. A. Methods Enzymol. 1987, 155, p335.
(4) Verma, S., and Eckstein, F. Annu. Rev. Biochem. 1998, 67, p99.
(5) Tyagi, S., and Kramer, F. R. Nat. Biotechnol. 1996, 14, p303.
(6) (a) Fodor, S. P., Read, J. L., Pirrung, M. C., Stryer, L., Lu, A. T., and Solas, D. Science 1991, 251, p767. (b) Schena, M., Shalon, D., Davis, R.

W., Brown, P. O. Science 1995, 270, p467.
(7) (a) Smith, L. M., Fung, S., Hunkapiller, M. W., Hunkapiller, T. J., Hood, L. E. Nucleic Acids Res. 1985, 13, p2399. (b) Kahl, J. D., and Greenberg, M. M. J. Am. Chem. Soc. 1999, 121, p597. (c) Dey, S., and Sheppard, T. L. Org. Lett. 2001, 3, p3983.
(8) Chehab, F. F.; Kan, Y. W. Proc. Natl. Acad. Sci.

U.S.A. 1989, 86, 9178.
(9) (a) Adamczyk, M., Chan, C. M., Fino, J. R., and Mattingly, P. G. J. Org. Chem. 2000, 65, p596. (b) Lyttle, M. H., Walton, T. A., Dick, D. J., Carter, T. G., Beckman, J. H., Cook, R. M. Bioconjugate Chem. 2002, 13, p1146. (c) Theisen, P., McCollum, C., Upadhya, K., Jacobson, K., Vu, H., and Andrus, A. Tetrahedron Lett. 1992, 33, p5033.
(10) (a) Saxon, K. E., and Bertozzi, C. R. Science 2000, 287, p2007. (b) Kristi, L., Saxon, K. E., Tirrell, D. A., and Bertozzi, C. R. Proc. Natl.

Acad. Sci. U.S.A. 2002, 99, p19.
(11) Yousaf, M. N., and Marksich, M. J. Am. Chem. Soc. 1999, 121, p4286.
(12) Kolb, H. C., Finn, M. G., and Sharpless, K. B. Angew. Chem. Int. Ed. 2001, 40, p2005.
(13) (a) Lewis, W. G., Green, L. G., Grynszpan, F., Radic, Z., Carlier, P. R., Taylor, P., Finn, M.

G., and Sharpless, K. B. Angew. Chem. Int. Ed. 2002, 41, p1053. (b) Huisgen, R. Pure Appl. Chem. 1989, 61, p613.
(14) (a) Mock, W. L., Irra, T. A., Wepsiec, J. P., and Manimaran, T. L. J. Org. Chem. 1983, 48, p3619. (b) Mock, W. L., Irra, T. A., Wepsiec, J. P., and Adhya, M. J. Org. Chem. 1989, 54, p5302. (c) Mock, W. L. Top. Curr. Chem. 1995, 175, p1.
(15) (a) Krasia, T. C., and Steinke, J. H. Chem.

Commun. 2002, p22. (b) Tuncel, D., and Steinke, J.

H. G. Chem. Commun. 2002, p496. (c) Tuncel, D., and Steinke, J. H. G. Chem. Commun. 2001, p253.
(16) Palacios, F., Retana, A. M., and Ragalday, J.

Heterocycles 1994, 38, p95.
(17) Sanger, F., Nicklen, S., and Coulson, A. R. Proc.

Natl. Acad. Sci. U.S.A. 1977, 74, p5463.
(18) McGeary, R. P. Tetrahedron Lett. 1998, 39, p3319.
(19) Ju, J. Anal. Biochem. 2002, 309, p35.
(20) Kheterpal, I., and Mathies, R. A. Anal. Chem. 1999, 71, 31A.
(21) Tuncel, D., and Steinke, J. H. G. Chem. Commun. 2002, pp496-497.

1. A method for covalently affixing a biomolecule to a second molecule comprising contacting a biomolecule having an azido group covalently and operably affixed thereto with a second molecule having an alkynyl group covalently and operably affixed thereto under conditions permitting a 1,3-dipolar cycloaddition reaction to occur between the azido and alkynyl groups, thereby covalently affixing the biomolecule to the second molecule.

2. The method of claim 1, wherein the biomolecule is selected from the group consisting of a nucleic acid, a protein, a peptide, a carbohydrate, and a lipid.

3. The method of claim 2, wherein the biomolecule is DNA.

4-6. (Canceled)

7. The method of claim 1, wherein the second molecule is selected from the group consisting of a biomolecule, a fluorescent label, a radiolabeled molecule, a dye, a chromophore, an affinity label, and a dextran.

8. The method of claim 1, wherein the second molecule is selected from the group consisting of an antibody, biotin, streptavidin, and a metabolite.

9. The method of claim 1, wherein the biomolecule is immobilized.

10. The method of claim 1, wherein the second molecule is immobilized.

11. The method of claim 1, wherein neither the biomolecule nor the second molecule is immobilized.

12. (Canceled)

13. The method of claim 1, wherein the conditions permitting a 1,3-dipolar cycloaddition reaction to occur comprise contacting at room temperature.

14. The method of claim 13, further comprising contacting in the presence of an agent which catalyzes a 1,3-dipolar cycloaddition reaction.

15. (Canceled)

16. (Canceled)

17. A method for covalently affixing a biomolecule to a second molecule comprising contacting a biomolecule having an alkynyl group covalently and operably affixed thereto with a second molecule having an azido group covalently and operably affixed thereto under conditions permitting a 1,3-dipolar cycloaddition reaction to occur between the alkynyl and azido groups, thereby covalently affixing the biomolecule to the second molecule.

18. The method of claim 17, wherein the biomolecule is selected from the group consisting of a nucleic acid, a protein, a peptide, a carbohydrate, and a lipid.

19-32. (Canceled)

33. A method for covalently affixing a biomolecule to a solid surface comprising contacting a biomolecule having an azido group covalently and operably affixed thereto with a solid surface having an alkynyl group operably affixed thereto under conditions permitting a 1,3-dipolar cycloaddition reaction to occur between the azido and alkynyl groups, thereby covalently affixing the biomolecule to the solid surface.

34. The method of claim 33, wherein the biomolecule is selected from the group consisting of a nucleic acid, a protein, a peptide, a carbohydrate, and a lipid.

35. The method of claim 34, wherein the biomolecule is DNA.

36-38. (Canceled)

39. The method of claim 33, wherein the solid surface is selected from the group consisting of glass, silica, diamond, quartz, gold, silver, metal, polypropylene, and plastic.

40. (Canceled)

41. The method of claim 39, wherein the solid surface is present on a bead, a chip, a wafer, a filter, a fiber, a porous media, or a column.

42. (Canceled)

43. The method of claim 33, wherein the conditions permitting a 1,3-dipolar cycloaddition reaction to occur comprise contacting at room temperature.

44. The method of claim 43, further comprising contacting in the presence of an agent which catalyzes a 1,3-dipolar cycloaddition reaction.

45. (Canceled)

46. (Canceled)

47. A method for covalently affixing a biomolecule to a solid surface comprising contacting a biomolecule having an alkynyl group covalently and operably affixed thereto with a solid surface having an azido group operably affixed thereto under conditions permitting a 1,3-dipolar cycloaddition reaction to occur between the alkynyl and azido groups, thereby covalently affixing the biomolecule to the solid surface.

48-80. (Canceled)
